SEQUENCE LISTING (23 sequences)

SEQ ID NO: 1 - DNA, 21 bp, Artificial Sequence
  Synthesized in the lab
gcccacagtcagattgaaaag

SEQ ID NO: 2 - DNA, 21 bp, Artificial Sequence
  Synthesized in the lab
cccacatcagctcattaacaa

SEQ ID NO: 3 - DNA, 20 bp, Artificial Sequence
  Synthesized in the lab
gcctgaagaacgagatcagc

SEQ ID NO: 4 - DNA, 20 bp, Artificial Sequence
  Synthesized in the lab
gtctgtgatgcccttagatg

SEQ ID NO: 5 - DNA, 20 bp, Artificial Sequence
  Synthesized in the lab
agcttatgacccgcacttac

SEQ ID NO: 6 - DNA, 22 bp, Artificial Sequence
  Synthesized in the lab
tgaccctcatggaaggttagaa

SEQ ID NO: 7 - DNA, 21 bp, Artificial Sequence
  Synthesized in the lab
ggacattgcattgcatgttgg

SEQ ID NO: 8 - DNA, 23 bp, Artificial Sequence
  Synthesized in the lab
atggtgtttaccgtaagctgtag

SEQ ID NO: 9 - DNA, 20 bp, Artificial Sequence
  Synthesized in the lab
ctcgcgttaccaggaagcat

SEQ ID NO: 10 - DNA, 17 bp, Artificial Sequence
  Synthesized in the lab
cagctgggccggttttg

SEQ ID NO: 11 - DNA, 20 bp, Artificial Sequence
  Synthesized in the lab
cactttatggcgacccaaca

SEQ ID NO: 12 - DNA, 20 bp, Artificial Sequence
  Synthesized in the lab
gcatcaacaggtggcgattt

SEQ ID NO: 13 - DNA, 25 bp, Artificial Sequence
  Synthesized in the lab
taattttcgtagattggcatcaaga

SEQ ID NO: 14 - DNA, 25 bp, Artificial Sequence
  Synthesized in the lab
ggacatgttcagggatcgccaggcg

SEQ ID NO: 15 - DNA, 23 bp, Artificial Sequence
  Synthesized in the lab
cgacgatgaagcatgtttagctg

SEQ ID NO: 16 - DNA, 24 bp, Artificial Sequence
  Synthesized in the lab
actggaagtaactttatcaaactg

SEQ ID NO: 17 - DNA, 22 bp, Artificial Sequence
  Synthesized in the lab
ctgaccaacttgctaacaatta

SEQ ID NO: 18 - DNA, 23 bp, Artificial Sequence
  Synthesized in the lab
gcggtctggcagtaaaaactatc

SEQ ID NO: 19 - DNA, 23 bp, Artificial Sequence
  Synthesized in the lab
gtgaaacagcattgctgtcactt

SEQ ID NO: 20 - DNA, 24 bp, Artificial Sequence
  Synthesized in the lab
ctaggccacagaattgaaagatct

SEQ ID NO: 21 - DNA, 25 bp, Artificial Sequence
  Synthesized in the lab
gtaggtggaaattctagcatcatcc

SEQ ID NO: 22 - DNA, 1878 bp, Homo sapiens
gcccacagtcagattgaaaagaggcgtcgggacaaaatgaacagtttcattgatgaattggcttctttggtaccaacatgcaatgcaatgtccaggaagttagataaactcaccgtgctaaggatggctgttcagcacatgaaaactttgagaggtaagagctcaggctttattgcctatatgcccttgactacaggtatgaccactcttgcctacactgtccctgacacaaatgttgatttctttcaacacgtaaccctaagtgacaggcctgtacatcttggcagaagatagggggccgaatcaggttgttaatgagctgatgtgggtattaagatgtcatttgcactaatacttccccagagcagataagcagatgagctcagatacttctctgtgtacatagcatatatacattgacatattcctgtttaccaaagaggacagacacctctcatgtcctcttgcctgtgcctggatcccaccccttgcaatgatcaaaatcaagaaatgtgggcgcacacagggaggcaggaaagagcctccaacttctaagagaaaaacttaccctggttcctctgatgaatgagtcacaggtggagagttgtatcatcagaaatgaacattgatgatattttcatcttcttggaattttcccgaactagattgactctctgtgagggaggcagtattttccttctttgctaagtgtggggagggtgagaaaacagaggcaggaaactggaagtaactttatcaaactgcaggaggaatctgaaatacatcacccattttggtgaatgtcgttcctggaattttccagttagtcttgggctttgggacaaacttgggaagggtgcttggccccatatgacctacatggtgagagaggcatccctctgtgtctgtataaggtcactagaaagaagttgcttttgttctgacttttgatcactattgagcaatacagaaacattgactctcatgcttgaagagtgatactgaaaatgggattttagtactgaagagatgggttataattaattaattgttagcaagttggtcagaaaatttcaagggatgtagattaagacatgtgccttaagctgggtgtggtggcacacgcctttaatcccagcacacaggaggcagaggcaggcggatttctgagttcgaggccagcctggtctacaaagtgagttccaggacagccagggctataaagagaaaccctgtctcgaaaaaaacaaaaaaacaaaacaaaacaaaacaaaacaaacaaacaacccccccaaaaaataacccaccaccaacaaaacatgtgccttgtgccttgttagctgatgtgaaaagtaccctggtaccctggattacaggtaggcttcatccgagcccttcactgggctgctctgcatgttcctgtcacttgcagctggcacttggctttgctaagctattgctcttgagaatttcggatcaaatgccttttaaaaaagtgtggtcccctcagtcctgaggctgaatcgtggctgagaatagcatgtcccccaggtcgccaagcttattaacttgcccagggaagtggacccagggcctggcagcttgggtgacttctctagaagctgtgaagtggggactggacagattggggatcagcctgttcactctggacttcagaacctgcccgggagactaaagagctgtggtcttttgtggggtttatagcatctcacagtatagctctggctagcctaaaactcactgtgtagaccaggctcaccttaatctcagagatctgcctacctcagtctccctagtgctggggttgaaggtgtgagccaccacgcctgttctctgatcaccgagtctctcgtgggc

SEQ ID NO: 23 - DNA, 6913 bp, Artificial Sequence
  Synthesized in the lab
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcccacagtcagattgaaaagaggcgtcgggacaaaatgaacagtttcattgatgaattggcttctttggtaccaacatgcaatgcaatgtccaggaagttagataaactcaccgtgctaaggatggctgttcagcacatgaaaactttgagaggtaagagctcaggctttattgcctatatgcccttgactacaggtatgaccactcttgcctacactgtccctgacacaaatgttgatttctttcaacacgtaaccctaagtgacaggcctgtacatcttggcagaagatagggggccgaatcaggttgttaatgagctgatgtgggtattaagatgtcatttgcactaatacttccccagagcagataagcagatgagctcagatacttctctgtgtacatagcatatatacattgacatattcctgtttaccaaagaggacagacacctctcatgtcctcttgcctgtgcctggatcccaccccttgcaatgatcaaaatcaagaaatgtgggcgcacacagggaggcaggaaagagcctccaacttctaagagaaaaacttaccctggttcctctgatgaatgagtcacaggtggagagttgtatcatcagaaatgaacattgatgatattttcatcttcttggaattttcccgaactagattgactctctgtgagggaggcagtattttccttctttgctaagtgtggggagggtgagaaaacagaggcaggaaactggaagtaactttatcaaactgcaggaggaatctgaaatacatcacccattttggtgaatgtcgttcctggaattttccagttagtcttgggctttgggacaaacttgggaagggtgcttggccccatatgacctacatggtgagagaggcatccctctgtgtctgtataaggtcactagaaagaagttgcttttgttctgacttttgatcactattgagcaatacagaaacattgactctcatgcttgaagagtgatactgaaaatgggattttagtactgaagagatgggttataattaattaattgttagcaagttggtcagaaaatttcaagggatgtagattaagacatgtgccttaagctgggtgtggtggcacacgcctttaatcccagcacacaggaggcagaggcaggcggatttctgagttcgaggccagcctggtctacaaagtgagttccaggacagccagggctataaagagaaaccctgtctcgaaaaaaacaaaaaaacaaaacaaaacaaaacaaaacaaacaaacaacccccccaaaaaataacccaccaccaacaaaacatgtgccttgtgccttgttagctgatgtgaaaagtaccctggtaccctggattacaggtaggcttcatccgagcccttcactgggctgctctgcatgttcctgtcacttgcagctggcacttggctttgctaagctattgctcttgagaatttcggatcaaatgccttttaaaaaagtgtggtcccctcagtcctgaggctgaatcgtggctgagaatagcatgtcccccaggtcgccaagcttattaacttgcccagggaagtggacccagggcctggcagcttgggtgacttctctagaagctgtgaagtggggactggacagattggggatcagcctgttcactctggacttcagaacctgcccgggagactaaagagctgtggtcttttgtggggtttatagcatctcacagtatagctctggctagcctaaaactcactgtgtagaccaggctcaccttaatctcagagatctgcctacctcagtctccctagtgctggggttgaaggtgtgagccaccacgcctgttctctgatcaccgagtctctcgtgggcaggaacccctagtgatggagttggccactccctctctgcgcgctcgctcgctcactgaggccgggcgaccaaaggtcgcccgacgcccgggctttgcccgggcggcctcagtgagcgagcgagcgcgcaggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

(19) United States
(12) Patent Application Publication
(10) Pub. No.: US 2023/0309522 A1
(43) Pub. Date: Oct. 5, 2023

(54) ANIMAL MODEL, METHODS FOR MAKING AND USING THEREOF, AND COMPOSITION FOR TREATING ATAXIA

(71) Applicant: City University of Hong Kong, Hong Kong (CN)

(72) Inventors: Sungchil YANG, Hong Kong (CN); Eric Hong Jian WONG, Hong Kong (CN)

(21) Appl. No.: 17/657,646

(22) Filed: Apr. 1, 2022

(51) Int. Cl. A01K 67/027; G01N 33/50; A61K 49/00; G01N 33/68; C12N 15/85; A61K 31/7088; A61P 25/28

(52) U.S. Cl. CPC A01K 67/0276; G01N 33/5088; A61K 49/0008; G01N 33/6896; C12N 15/8509; A61K 31/7088; A01K 2217/054; C12N 2015/8536; A01K 2217/15; G01N 2333/4727; A01K 2227/105; A01K 2267/0318; A61P 25/28

(57) ABSTRACT

The present invention provides a platform for in vitro or in vivo study of the correlation between a Purkinje cell-specific, circadian clock gene and ataxia, in particular, a non-human transgenic animal model induced by genetic modification to knockdown the circadian clock gene, Bmal1, which causes abnormal diurnality and loss of certain motor skills and learning ability in a subject. The present invention also relates to methods of making and using the platform for various applications. A composition including a vector carrying the Bmal1 gene for restoring expression thereof in the subject's cerebellum to potentially treat ataxia arising from the Bmal1 gene deficiency is also provided.

### TECHNICAL FIELD

The present invention relates to an animal model for ataxia, in particular, a circadian clock gene-deficient animal model having ataxia-like symptoms and behaviours, and methods for making and using thereof. The present invention also relates to a composition including a nucleotide sequence of the circadian clock gene for restoring expression of the circadian clock gene in a recipient devoid of the same.

### BACKGROUND

Ataxia is largely caused by the malfunction of the vestibular or proprioceptive afferent circuits which often leads to cerebellar impairment 1. This neurological disorder is involved in the impaired coordination of voluntary muscle movements such as abnormal gait, balance, and postural control^{1,2}. Ataxia is worldwide reported secondary to stroke for the age after 65 while it attains a peak in young adulthood³. To date, the prevalence of ataxia patients is projecting to a steep increment in population^{3,4}. Ataxia is generally categorized into two major groups that are non-hereditary and hereditary ataxia. For example, sensory ataxia as non-hereditary ataxia is induced by the proprioceptive injury of the posterior columns of the spinal cord or peripheral nerves^{5,6}. On the other hand, spinocerebellar ataxia (SCA) in which cerebellar dysfunction is a major cause is classified to be hereditary ataxia^{7,8}. Most SCAs are caused by prominent damage to cerebellar Purkinje neurons which is a root cause for cerebellar dysfunction⁹.

Circadian clock genes such as Per and Cry are suggested to be involved in an inherited form of ataxia^{10,11}. As another circadian clock gene, aryl hydrocarbon receptor nuclear translocator-like protein 1 (ARNTL) or brain and muscle ARNT-like protein 1 (Bmal1) gene has been widely reported in controlling the action of circadian rhythm but not identified as a genetic etiology of ataxia¹². Circadian rhythm is driven by rhythmic expression of a large fraction of the transcriptome in the regulation of biochemical, physiological and behavior functions¹³. A transcriptional-translational negative feedback loop with a set of core clock genes requires the time-locked expression of circadian clock genes^{13,14}. Bmal1 is one of two master heterodimer transcription factors with Clock^{15,16}. These heterodimers rhythmically activate the transcriptional repressors of Per and Cry¹⁷. Previous studies reported that genetic deletion of Bmal1 ablates the regulation of circadian rhythm, having catastrophic effects on the sleep cycle such as reduced sleep efficiency, reduced rapid eye movement sleep, increased non-rapid eye movement sleep and inability to recover from sleep deprivation^{15,18}. To date, a causal relationship of Bmal1 to ataxia is yet to be established.

There is a need for a model or platform to verify their relationship, and based on this relationship to develop a composition for treating ataxia or related diseases, conditions, or symptoms associated with the deficiency of Bmal1.

### SUMMARY OF THE INVENTION

Accordingly, one of the objectives of the present invention is to develop an animal model with a whole body knock-out of Bmal1 to verify the relationship between a deficiency of Bmal1 genes in cerebellum and ataxia. This animal model may be used in different applications such as drug screening for potential candidates of treating cerebellar ataxia due to Bmal1 gene-deficiency. A nucleic acid, vector or composition containing the genes associated with the ataxia is also provided for a recipient devoid of the same to restore the expression level thereof such that a circadian rhythm and associated motor skills of the recipient can be resumed.

Therefore, the present invention provides a non-human animal model for verifying the relationship between Bmal1 and ataxia.

In an exemplary embodiment, the present animal model is devoid of expression of Bmal1, leading to a loss of diurnality.

In certain embodiment, the devoid of Bmal1 expression in the animal model can be chemically-induced or genetically-modified.

In one embodiment, the genetically-modified animal model shows similar behaviours to those known in the chemically-induced animal model.

In another embodiment, the chemically-induced animal model of the present invention shows no expression of Bmal1 by a systemic administration of an ataxia-inducing agent.

In certain embodiments, the ataxia-inducing agent is selected from a cerebellar ataxia-inducing agent.

In a preferred embodiment, the cerebellar ataxia-inducing agent is selected from 3-acetyl pyridine (3AP).

In certain embodiments, the genetically modified animal model of the present invention is a whole body Bmal1-knockout (KO) mouse model.

In other embodiments, the genetically modified animal model is a Purkinje cell-specific Bmal1 knockout (PCP-Bmal1 KO) mouse model.

In a first aspect, a non-human animal model having a Purkinje cell-specific gene deficiency leading to ataxia is provided, wherein the Purkinje cell-specific gene is one of the circadian clock genes relating to regulation of circadian rhythm, motor skills and learning ability of a subject.

In certain embodiments, the Purkinje cell-specific gene is brain and muscle ARNT-like protein 1 (Bmal1) gene.

In certain embodiments, the devoid of expression of Bmal1 in the animal model leads to a loss of diurnality.

In certain embodiments, the subject is human.

In certain embodiments, the animal model shows similar behaviours to those known in a comparable, chemically-induced ataxia animal model.

In certain embodiments, the comparable, chemically-induced ataxia animal model is provided by a systemic administration of an ataxia-inducing agent to an animal of the same species as the present animal model.

In certain embodiments, the ataxia-inducing agent is selected from a cerebellar ataxia-inducing agent.

In one embodiment, the cerebellar ataxia-inducing agent is selected from 3-acetyl pyridine (3AP).

In an exemplary embodiment, the animal model is a knockout mouse model.

In a second aspect, the present invention provides a method of screening compounds or molecules that are capable to restore circadian rhythm, motor skills and learning ability in a subject whose loss of the circadian rhythm, motor skills and learning ability is due to a Purkinje cell-specific gene deficiency, where the method includes the steps of:
providing the animal model described herein which includes a knockdown of the Purkinje cell-specific gene being one of the circadian clock genes relating to regulation of the circadian rhythm, motor skills and learning ability of the subject;
introducing a composition that is known to be capable to restore the circadian rhythm, motor skills and learning ability of the subject into a first population of the animal model;
introducing the compounds or molecules into a second population of the animal model;
obtaining a sample from each of the first population and the second population of the animal model; and
comparing expression level of the Purkinje cell-specific gene or a biomarker thereof in the sample of the first population with that in the sample of the second population,
if the expression level of the Purkinje cell-specific gene or the biomarker thereof in the second population is comparable to that in the first population, the compounds or molecules will be determined to be capable to restore the circadian rhythm, motor skills and learning ability of the subject; and
the composition contains the Purkinje cell-specific gene and is capable to restore expression of the Purkinje cell-specific gene specifically in Purkinje cells and cerebellum of the subject.

In certain embodiments, the composition is introduced either locally or systematically into the animal model.

In certain embodiments, the composition is or includes a viral vector containing the Purkinje cell-specific gene.

In one embodiment, the viral vector is selected from adeno-associated virus.

The method of the second aspect may further include the steps of:
performing motor skill and learning ability related behavioral tests on the first and second populations of the animal model before and after being respectively introduced with the composition and the compounds or molecules, and
comparing the behavioral test results with those obtained from animals of the same species as the animal model but without the Purkinje cell-specific gene deficiency.

In certain embodiments, the behavioral tests include at least four tests which are footprint test, irregular ladder test, rotarod test and body balance test.

In certain embodiments, the sample obtained from the first and second populations of the animal model includes cerebellar tissues and at least one type of nucleic acid from the Purkinje cells of the animal model.

In certain embodiments, the at least one type of nucleic acid is a total RNA of the Purkinje cells.

In certain embodiments, the cerebellar tissues are subject to immunohistochemical analysis to detect the biomarker for Purkinje cells.

In an exemplary embodiment, the Purkinje cell-specific gene is brain and muscle ARNT-like protein 1 (Bmal1) gene.

In certain embodiments, the biomarker is calbindin.

In a further embodiment, the number of Purkinje cells expressing calbindin corresponds to the expression level of Bmal1 gene in the sample.

In certain embodiments, the expression level of Bmal1 gene is determined by expression level of messenger RNA (mRNA) thereof from the total RNA obtained from Purkinje cells or corresponding cerebellar tissues.

In certain embodiments, the subject is human.

In a third aspect, the present invention provides a method for making the non-human animal model described herein, comprising crossbreeding a target mouse having a locus with a cell-specific Cre recombinase expressed mouse, where the locus contains the Bmal1 gene floxed with at least two loxP sites; the expressed Cre recombinase specifically targets Purkinje cells (or responds to Purkinje cell-specific protein) and recognizes the at least two loxP sites in order to delete the locus containing the Bmal1 gene in the Purkinje cells of the target mouse, such that a Purkinje cell-specific Bmal1 knockout (PCP-Bmal1 KO) mouse is generated.

In a fourth aspect, the present invention provides a composition including a Purkinje cell-specific gene relating to regulation of circadian rhythm, motor skills and learning ability of a subject, where the Purkinje cell-specific gene is one of the circadian clock genes and the expression thereof is higher at daytime than nighttime.

In an exemplary embodiment, the composition is selected from a nucleic acid or viral vector capable of restoring expression of the Purkinje cell-specific gene in the subject with a deficiency of the Purkinje cell-specific gene after administering the composition locally or systematically to the cerebellum of the subject.

In one embodiment, the viral vector is selected from adeno-associated virus in different serotypes including, but not limited to, serotype 2 (or abbreviated as AAV2).

In certain embodiments, the Purkinje cell-specific gene is Bmal1.

In certain embodiments, the local administration of the composition includes stereotaxic local injection to cerebellum of the subject.

In certain embodiments, the systematic administration of the composition includes intraperitoneal (i.p.) injection to systemic circulation of the subject.

In a fifth aspect, the present invention provides a method for treating ataxia in a subject in need thereof, where the method includes administering the composition described herein locally or systematically to the cerebellum of the subject.

In certain embodiments, the composition includes a viral vector containing the Purkinje cell-specific gene to restore expression of the gene in the subject devoid of Bmal1 expression.

In certain embodiments, the subject is Bmal1-gene deficient.

In certain embodiments, the subject includes human and non-human animals with deficiency of Bmal1 gene.

In certain embodiments, the subject expresses ataxia-like symptoms similar to those known induced by a cerebellar ataxia inducing agent.

In certain embodiments, the cerebellar ataxia inducing agent is 3-acetyl pyridine.

Other aspects of the present invention include a method for restoring Bmal1 expression in a Bmal1-deficient animal; a method and platform (in vitro or in vivo) for screening potential drug candidates to prevent, treat, or alleviate disease, conditions or symptoms arising from or similar to ataxia due to deficits of Bmal1 expression; a therapeutic regimen including the use of a viral vector containing the Bmal1 gene to be administered alone or in combination with any other compounds or molecules to a subject in need thereof for treating ataxia.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1A schematically depicts a footprint test (one of the behavioral tests) on animal models prepared according to certain embodiments of the present invention; error bars indicate the SEM; **p-value<0.01; ***p-value<0.001;

FIG. 1B schematically depicts an irregular ladder test (one of the behavioral tests) on an animal model prepared according to certain embodiments of the present invention; error bars indicate the SEM; **p-value<0.01; ***p-value<0.001;

FIG. 1C schematically depicts a rotarod test (one of the behavioral tests) on animal models prepared according to certain embodiments of the present invention; error bars indicate the SEM; **p-value<0.01; ***p-value<0.001;

FIG. 1D schematically depicts an elevated beam test (one of the behavioral tests) on animal models prepared according to certain embodiments of the present invention; error bars indicate the SEM; **p-value<0.01; ***p-value<0.001;

FIG. 2A shows the results of the rotarod test on different animal models including chemically-induced ataxia animal model (local 3AP) and genetically-modified ataxia animal model (PCP-Bmal1 KO) as depicted in FIG. 1C;

FIG. 2B shows the results of the elevated beam test on different animal models as depicted in FIG. 1D;

FIG. 2C shows an average number of slips measured on the chemically-induced ataxia model during the elevated beam test depicted in FIG. 1D as compared to that measured on the positive control (wildtype animal);

FIG. 2D shows an average number of slips measured on the genetically-modified ataxia animal model during the elevated beam test depicted in FIG. 1D;

FIG. 3A shows Bmal1 expression pattern in a cerebellum of a wildtype animal in a day and night cycle;

FIG. 3B shows mRNA expression of various circadian clock genes in cerebellums from different animal models including the chemically-induced (local 3AP) and genetically-modified (PCP-Bmal1 KO) ataxia animal models prepared according to certain embodiments of the present invention as compared to the controls (wildtype and Bmal1 cre);

FIG. 3C shows immunohistochemistry images of cerebellar tissues from the chemically-induced (3AP-injected), genetically-modified (PCP-Bmal1 KO) ataxia animal models prepared according to certain embodiments of the present invention, and a control (wildtype) animal; arrows indicate the location of Purkinje cells on the tissue samples of different animal models; insets in the image of the tissue sample from the wildtype animal's cerebellum show the location of Purkinje cells in a higher magnification (10×); calbindin (red) as a molecular marker of Purkinje cells; DAPI stain (blue) for normalization; ML: molecular layer; PC: Purkinje cells; GC: granular cells;

FIG. 3D shows expression level of calbindin in different cerebellar tissue samples from immunohistochemistry data obtained from the images shown in FIG. 3C; error bars indicate the SEM; ***p<0.001; n.s: not significant;

FIG. 4A schematically depicts a design of a Bmal1-containing viral vector (left panel) and site specific administration of the viral vector into the genetically-modified ataxia animal model (right panel) prepared according to certain embodiments of the present invention;

FIG. 4B shows a timeline for a series of tests on two ataxia animal models prepared according to certain embodiments of the present invention;

FIG. 4C shows hind paw distance measured in the footprint test on different animal models introduced with or without the viral vector as shown in FIG. 4A.

FIG. 4D shows stride distance measured in the footprint test on different animal models introduced with or without the viral vector as shown in FIG. 4A;

FIG. 4E shows front paw distance in the footprint test depicted in FIG. 1A on different animal models introduced with or without the viral vector as shown in FIG. 4A;

FIG. 4F shows an average number of slips in the irregular ladder test depicted in FIG. 1B on different animal models introduced with or without the viral vector as shown in FIG. 4A;

FIG. 4G shows walking performance in the rotarod test depicted in FIG. 1C on different animal models introduced with or without the viral vector as shown in FIG. 4A;

FIG. 4H shows an average number of slips in the elevated beam test depicted in FIG. 1D on different animal models introduced with or without the viral vector as shown in FIG. 4A;

FIG. 4I shows immunohistochemistry images of cerebellar tissue samples from different animal models after introduced with the viral vector as shown in FIG. 4A; insets in each of the images in the middle column shows calbindin expression (red) in a higher magnification (10×); DAPI stain (blue) for normalization; ML: molecular layer; PC: Purkinje cells; GC: granular cells;

FIG. 4J shows calbindin expression level in different tissue samples from the animal models after introduced with the viral vector as shown in FIG. 4A; error bars indicate the SEM; **p-value<0.01; ***p-value<0.001;

FIG. 4K shows mRNA expression level of Bmal1 in different animal models after introduced with the viral vector as shown in FIG. 4A; error bars indicate the SEM; **p-value<0.01; ***p-value<0.001;

FIG. 5A schematically depicts a neural circuitry for motor control in a typical cerebellum of an animal comparable to a wildtype animal of the present animal model;

FIG. 5B shows relative mRNA expression level of various circadian clock genes in motor cortex of the cerebellums from different animal models prepared according to certain embodiments of the present invention after introduced with the viral vector as shown in FIG. 4A; each animal model: n=8; error bar indicates the SEM; p-value>0.05:

FIG. 5C shows relative mRNA expression level of various circadian clock genes in inferior olive of the cerebellums from different animal models prepared according to certain embodiments of the present invention after introduced with the viral vector as shown in FIG. 4A; each animal model: n=8; error bar indicates the SEM; p-value>0.05;

FIG. 5D shows relative mRNA expression level of various circadian clock genes in striatum of the cerebellums from different animal models prepared according to certain embodiments of the present invention after introduced with the viral vector as shown in FIG. 4A; each animal model: n=8; error bar indicates the SEM; p-value>0.05;

FIG. 6A shows immunohistochemistry images of tissue samples from substantial nigra compacta (SNc) of different animal models prepared according to certain embodiments of the present invention, indicating expression of Tyrosine Hydrolase (TH) (red) as a molecular marker of dopamine neuron; DAPI for normalization (blue); error bars indicate the SEM: p>0.05;

FIG. 6B shows relative TH expression level in SNc of different animal models obtained from immunohistochemistry data of different tissue samples as shown in FIG. 6A;

FIG. 7 schematically depicts the design of a Bmal1-containing viral vector (pAAV-CMV-GFP Bmal1) according to certain embodiments of the present invention;

FIG. 8 shows other circadian clock gene mRNA expression than Bmal1 in different animal models after local administration of a viral vector containing Bmal1.

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

To better understand the relationship between Bmal1 with ataxia, two Bmal1-deficient mice, each having chemical or genetic antagonism^{15,16}, are tested. It is found that genetic Bmal1 deficient mice show ataxia-like behaviors similar to those already known in 3-acetyl pyridine (3AP)-intoxicated ataxia mice. Additionally, 3AP-injected mice did not express Bmal1, which further demonstrates a close association of Bmal1 with ataxia. Interestingly, mice receiving chemical and genetic deletion of Bmal1 lost diurnality, showing no behavioral differences between daytime and nighttime. This behavioral abnormality disappeared in the viral expression of Bmal1 in cerebellar Purkinje cells, which suggests a causal relationship between Bmal1 and ataxia. These findings also provide an insight to develop a therapeutic regimen, such as a gene therapy, to treat ataxia associated with loss of this circadian clock gene by restoring the expression of the same in cerebellar Purkinje cells or tissue of a recipient.

### EXAMPLE 1—ESTABLISHMENT OF ATAXIA ANIMAL MODELS

Six to seven-week-old C57BU6J and B6.129-Arnt1^{tm1Bra}/J (Bmal1 gene-deficient mice) male mice were used for all experiments described hereinafter. All experiments were performed on mice anesthetized by an intraperitoneal (i.p.) injection of 100 mg ketamine/kg and 20 mg xylazine/kg, based on their body weights.

In this example, a chemically-induced ataxia animal model was established upon systematic application of 3AP^{19,20}. Besides systematic (i.e. intraperitoneal, i.p., injection), local injection [in cerebellum, coordinate (Bregma): −7.08, 0, −2.1] of 3AP in C57BU6J mice could also be used to generate the chemically-induced ataxia animal models²³⁻²⁵. Initially, different concentrations of 3AP were applied for determining the least concentration of 3AP for life-long induction of ataxia-like behaviors (Table 1).

TABLE 1

| | | | |
|---|---|---|---|
| | | Number of | |
| Concentration | Survival time | animals used in | |
| (mg/kg) | (hours) | present disclosure | Reference |

Intraperitoneal systematic injection

| | | | |
|---|---|---|---|
| 70 | 0.25 | 3 | Wecker et al.²⁰ (2013) |
| 65 | 3 | 3 | Mohammadi et al.¹⁹ |
| | | | (2017) |
| 50 | 8 | 3 | Present disclosure |
| 45 | 24 | 3 | Present disclosure |
| 35 | >120 | >3 | Present disclosure |
| | | (for the study) | |
| 25 | No ataxia | 3 | Present disclosure |
| | observed | | |

Stereotaxic local injection to the cerebellum

| | | | |
|---|---|---|---|
| 0.2 | No survive | 3 | Present disclosure |
| 0.15 | No survive | 3 | Present disclosure |
| 0.1 | >120 | >3 | Present disclosure |
| | | (for the study) | |
| 0.05 | No ataxia | 3 | Present disclosure |
| | observed | | |

In addition to the chemically-induced ataxia model, a genetically-modified ataxia model, i.e., whole body Bmal1 knockout (KO) mice, was generated. To create a site specific Bmal1 in cerebellum of the KO mice, Bmal1^{flox+/+}-cre (Bmal1-cre) mouse was crossbred with B6.Cg-Tg (Pcp2-cre)3555Jdhu/J mouse (Purkinje cre recombinase express mouse). Bmal1 knockdown was verified though polymerase chain reaction (PCR). Ataxia-like behaviors were confirmed through various behavioral tests, the detail of which will be described hereinafter. All protocols were approved by the Institutional Animal Care and Use Committee of City University of Hong Kong. The current research was conducted in accordance with the guidelines from the university animal welfare committee and has complied with all the ethics policies.

### EXAMPLE 2—BEHAVIOR TESTS ON ANIMAL MODEL

Four different behavior tests were used in the present disclosure: footprint, irregular ladder, rotarod and body balance (elevated beam) tests. These behavior tests have been widely used for detection of ataxia⁴⁰⁻⁴². Two different time durations were used to differentiate the peak and trough expressions of the Bmal1 gene. i.e., daytime described herein refers to seven hours later after light exposure (˜2 pm); nighttime described herein refers to four hours later after dim light (˜1 pm). These behavior tests were carried out at daytime (˜2 pm) and nighttime (˜11 pm), given the differential expression of Bmal1 in cerebellum having the highest level at ˜2 pm and the lowest level at ˜11 pm.

Footprint test: Three measurements were included: i) front paw distance; ii) hind paw distance; and iii) stride. The footprint behavior tool was custom-made to have a narrow, foot-printable glass plate with the length of 85 cm and the width of 5 cm. Each paw distance was measured with footsteps printed during the walking across the narrow road.

Irregular ladder test: A mouse was requested to walk through an irregularly arranged ladder with a distance of 85 cm and a width of 5 cm. Every single slip during walk steps was counted as a failure of gait and balance measurement.

Rotarod test: It has been widely used in determining general motor skills. In brief, mice walked on the rotarod with a width of 5 cm, a height of 20 cm and a diameter of 3 cm of rolling rods. The total time for mice to sustain on the rotarod in increasing speeds at a unit of revolution per minute (rpm) was taken for a measurement of motor skills such as balance and gait functions. Motor learning was calculated as a percentage change in the performance (at a maximum speed) over 5 min, 10 min or 15 min blocks sequentially.

Elevated beam test: Every single slip was counted during the walking⁴³. Mice were requested to walk through a wall with a distance of 40 cm, a height of 16 cm and a width of 0.5 cm. All tests were conducted in a quiet environment without noise and bright light disturbance.

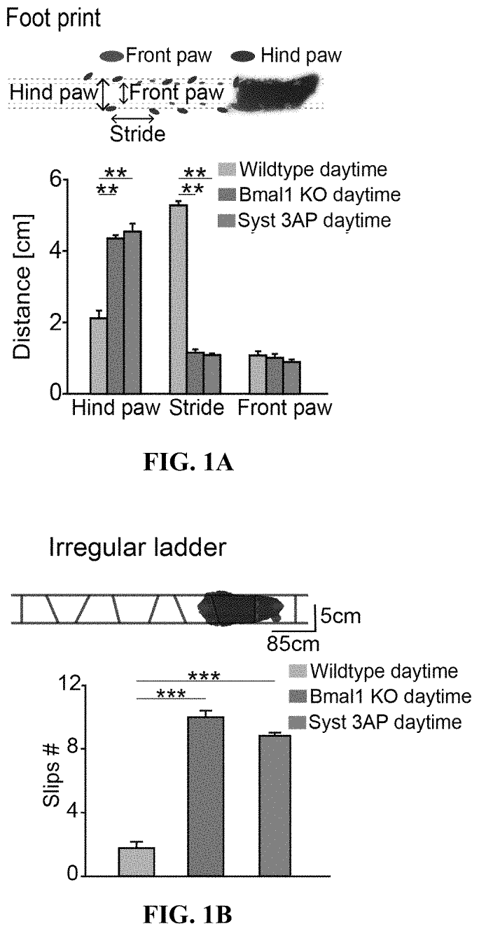

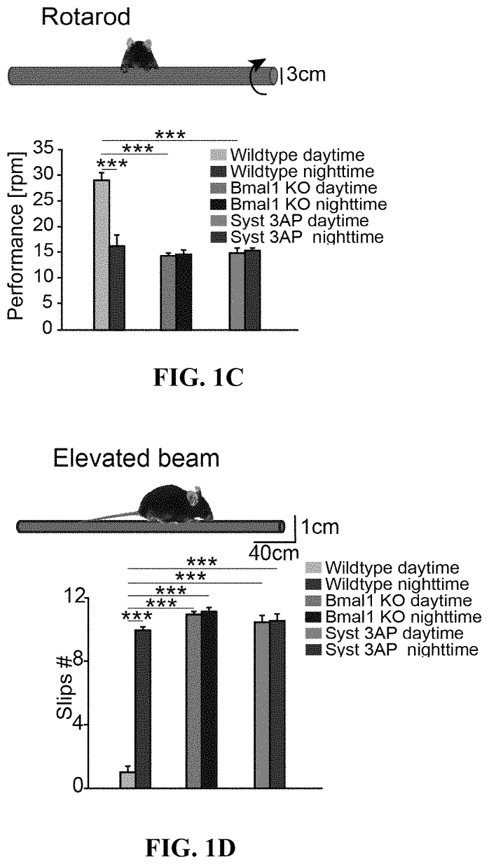

35 mg/kg 3AP-injected mice prepared according to Table 1 showed the least behavioral threshold of abnormal gait in the footprint task when they were requested to walk through a narrow and long road. There were significant differences in hind paw distance and stride, but not front paw distance, between 3AP-induced ataxia and wildtype mice (FIG. 1A, hind paw distance, WT, 2.09 cm±0.185 vs. 3AP, 4.67±0.208 cm, n=18, one-way ANOVA, p=0.0088, F=985.1; stride. WT, 5.27±0.118 cm vs. 3AP, 1.11±0.088 cm, p=0.0045. F=149.4; front paw distance. WT, 1.084±0.137 cm vs. 3AP, 0.832±0.112 cm, p=0.626, F=0.242). The same behavior assay was applied to whole-body Bmal1 knockout (KO) mice so as to identify a correlation of Bmal1 to ataxia. Bmal1 KO mice showed wider hind paw distance and narrower stride like 3AP-induced ataxia mice (hind paw distance, 4.32±0.082 cm, one-way ANOVA, n=15, p=0.0094. F=1262.8; stride, 1.18±0.123 cm, p=0.0075, F=7928.6; front paw distance, 0.908±0.102 7 cm, p=0.412, F=0.240). There were no differences in all of the three footprint measurements between 3AP-induced ataxia and Bmal1 KO mice (hind paw distance, p=0.472; front paw distance, p=0.612; stride, p=0.568). Also, the irregular ladder assay was performed for testing an ability of motor coordination (FIG. 1B). Both Bmal1 KO and 3AP-induced ataxia mice had significant difference against wildtype mice, showing increased number of slips during the walking along an irregular ladder (WT, 1.61±0.257 vs. 3AP, 8.92±0.178, n=18, p<0.001, F=397.9; WT vs. Bmal1 KO, 9.82±0.312, n=18, p<0.001, F=603.6).

Next, a rotarod task was set out to investigate if the uncoordinated movement is due to impaired motor skills in the two ataxia models and is circadian rhythm-dependent. In the two ataxia models (chemically-induced and genetically-modified ataxia mice), there was drastic reduction in the rotarod-based motor behavior of the wildtype at daytime compared to that at nighttime (FIG. 1C, WT day, 28.00±1.76 rpm vs. WT night, 17.25±2.08 rpm, p<0.001, F=288.8; WT day vs. 3AP day, 14.57±1.38 rpm, p<0.001, F=398.5; 3AP day vs. 3AP night, 15.22±0.78 rpm, p=0.805, F=0.063; WT day vs. Bmal1 KO day, 14.33±0.66 rpm, p<0.001, F=468.7; Bmal1 KO day vs. Bmal1 KO night, 14.67±0.57 rpm, p=0.786, F=0.077). Such behavioral impairment at nighttime was also observed in the elevated beam test when they were requested to walk along a narrow beam. The two ataxia models had the increased number of slips regardless of time shifting (FIG. 1D, WT day, 0.93±0.23 vs. WT night, 9.92±0.11, p<0.001, F=414.4; WT day vs. 3AP day, 10.23±0.23, p<0.001, F=351.6; 3AP day vs. 3AP night, 10.24±0.23, p=0.786, F=0.077; WT day vs. Bmal1 KO day, 10.92±0.10, p<0.001, F=700.0; Bmal1 KO day vs. Bmal1 KO night, 11.08±0.173, p=0.727, F=0.127). These behavioral observations indicate that the genetically-modified ataxia mice (Bmal1 KO mice) show significant impairments of motor coordination, balance, and motor skills, especially at daytime.

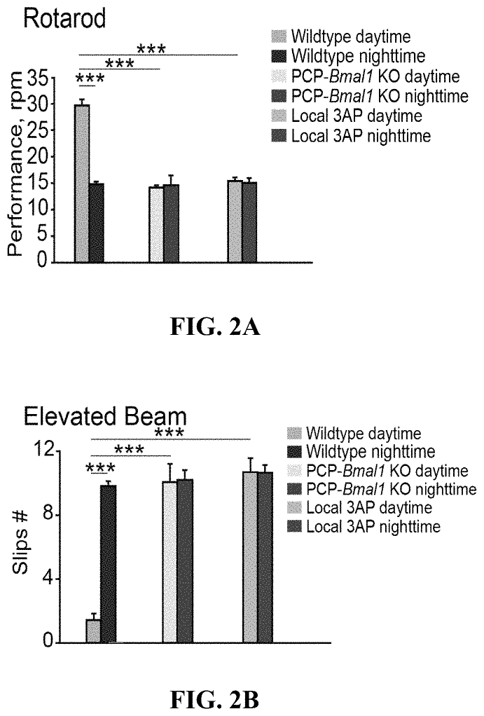

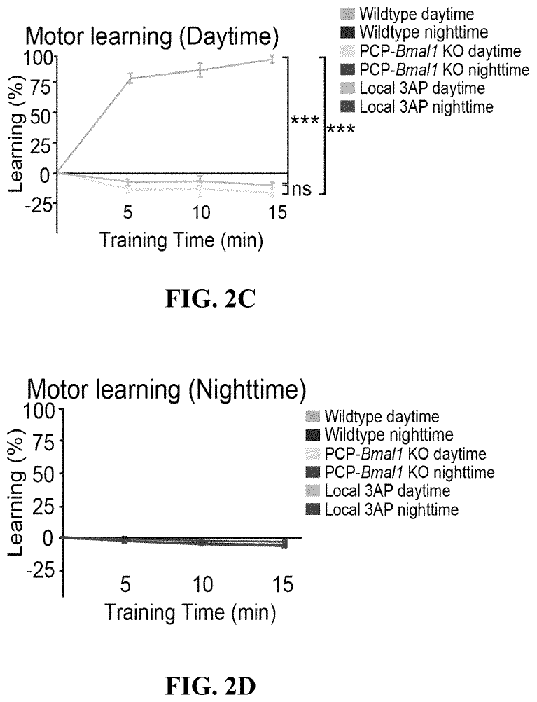

It was further investigated whether cerebellar elimination of Bmal1 causes the same motor and learning skills impairment like the whole-body Bmal1-deficiency mice. Motor coordination and skills of mice receiving 3AP local injection specifically in the cerebellum were examined with the rotarod and elevated beam tasks. Cerebellar intoxication with 3AP showed decreased performance in the rotarod test at daytime (FIG. 2A, Rotarod: WT, 29.32±0.96 rpm, n=12 vs. Local 3AP, 14.32±1.02 rpm, n=12, p<0.001, F=4234.2) and increased slips in the elevated beam behavior test (FIG. 2B, elevated beam: WT, 0.87±0.48 vs. Local 3AP, 10.46±0.92, n=12, p<0.001, F=2145.2). To further test the role of cerebellar Purkinje cells in Bmal1-deficient ataxia, Bmal1^{flox+/+}-cre mice were crossbred with B6.Cg-Tg (Pcp2-cre)3555Jdhu/J mice, named BAC-Pcp2-IRES-Cre mice. Like the local 3AP-injected mice, they had decreased performance in the rotarod behavior test and increased number of slips in the elevated beam test measured at daytime (Rotarod: WT vs. PCP-Bmal1 KO, 13.32±0.77 rpm, n=12, p<0.001, F=1615.7; elevated beam: PCP-Bmal1 KO, 9.86±1.23, n=12, p<0.001, F=691.9). Then, we tested whether cerebellar elimination of Bmal1 also impairs motor skills and learning. Both cerebellar 3AP-injected and PCP-Bmal1 KO mice had a significant decrease in motor learning during the rotarod behavior test only in the daytime, but not nighttime (FIG. 2C, All comparison of WT vs. 3AP or PCP-Bmal1 KO, p<0.001, Table 2 for the statistics in detail). These results demonstrate a critical role of Purkine cells' Bmal1 in regulating motor skills and learning.

TABLE 2

| Animal strain | | PC-Bmal1 KO | 3AP local |
|---|---|---|---|
| Training time (min) | Wild Type | (p-value) | (p-value) |
| 5 | 76.4 ± 7.21 | −11.9 ± 3.28% | −4.8 ± 4.22% |
| | | (p < 0.001) | (p < 0.001) |
| 10 | 88.6 ± 9.32 | −10.3 ± 8.26% | −4.1 ± 7.32% |
| | | (p < 0.001) | (p < 0.001) |
| 15 | 95.46 ± 4.44 | −11.2 ± 5.32% | −4.5 ± 4.12% |
| | | (p < 0.001) | (p < 0.001) |

Error bars indicated the SEM;
**p-value <0.01;
***p-value <0.001

### EXAMPLE 3—ABNORMAL CEREBELLAR BMAL1 EXPRESSION FOR ATAXIA

To verify Bmal1 gene in the animal model, DNA was extracted for genotyping by using DNA extraction kit (Themofisher scientific, USA). For the chemically-induced ataxia model. DNA was extracted before and after a systematic application of the 3AP. The presence of the Bmal1 gene was confirmed by end-point PCR using the Bmal1 primers (Table 3). The 20 μl reaction mixture for detection of Bmal1 gene included 75 ng genomic DNA, 2.0 μl forward and reverse primer, 2× Phire tissue direct PCR master mix (Takara, Japan) and sterile distilled water. The reaction had sequential processes: denaturation of the genomic DNA for an initial cycle at 98° C. for 5 minutes; 35 cycles of denaturation at 98° C. for 5 seconds; annealing at 53.9° C. for 5 seconds; elongation at 72° C. for 20 seconds; and termination at 72° C. for 1 minutes. For cre and lox genes verification, the reaction had sequential processes: denaturation of the genomic DNA for an initial cycle at 94° C. for 5 minutes; 28 cycles of denaturation at 94° C. for 1 min; annealing at 64° C. for 1 minute; elongation at 72° C. for 1 minute; and termination at 72° C. for 2 minutes. Following the PCR reaction, 2 μl products were loaded into the wells of a 1% agarose gel using a 6× loading buffer and electrophoresed for 45 minutes at 100V in 1×TBE buffer. A 100 bp DNA ladder molecular weight marker (Takara, Japan) was used for band size identification (Bio Rad).

TABLE 3

| Targeted | | | SEQ ID | |
|---|---|---|---|---|
| gene | Primer sequence (5′-3′) | | No | Remarks |
| Bmal1 | Forward: | GCCCACAGTCAGATTGAAAAG | 1 | Qualitative |
| | Reverse: | CCCACATCAGCTCATTAACAA | 2 | |
| | Reverse: | GCCTGAAGAACGAGATCAGC | 3 | |
| 18S | Forward: | GTCTGTGATGCCCTTAGATG | 4 | Quantitative |
| | Reverse: | AGCTTATGACCCGCACTTAC | 5 | |
| Arntl | Forward: | TGACCCTCATGGAAGGTTAGAA | 6 | |
| (Bmal1) | Reverse: | GGACATTGCATTGCATGTTGG | 7 | |
| Clock | Forward: | ATGGTGTTTACCGTAAGCTGTAG | 8 | |
| | Reverse: | CTCGCGTTACCAGGAAGCAT | 9 | |
| Per1 | Forward: | CAGCTGGGCCGGTTTTG | 10 | |
| | Reverse: | CACTTTATGGCGACCCAACA | 11 | |
| Cry1 | Forward: | GCATCAACAGGTGGCGATTT | 12 | |
| | Reverse: | TAATTTTCGTAGATTGGCATCAAGA | 13 | |
| PCP cre | Cre: | GGACATGTTCAGGGATCGCCAGGCG | 14 | Qualitative |
| | Cre-Beta: | CGACGATGAAGCATGTTTAGCTG | 15 | |
| | oIMR7525: | ACTGGAAGTAACTTTATCAAACTG | 16 | |
| | oIMR7526: | CTGACCAACTTGCTAACAATTA | 17 | |
| | olMR 1084: | GCGGTCTGGCAGTAAAAACTATC | 18 | |
| | olMR 1085: | GTGAAA CAGCATTGCTGTCACTT | 19 | |
| | olMR 7338: | CTAGGCCACAGAATTGAAAGATCT | 20 | |
| | olMR 7339: | GTAGGTGGAAATTCTAGCATCATCC | 21 | |

To prepare RNA by RT-PCR, mice were deeply anesthetized with isoflurane inhalation. After brain removal, RNA was collected from several brain regions associated with the motor coordination such as cerebellum, striatum, inferior olive and motor cortex. Total RNA was extracted by TRIzol reagent-based procedure (Thermofisher scientific, USA) and quantified by NanoDrop 2000 spectrophotometer (Bio-rad, USA). The total RNA obtained (20 μg) was reverse-transcribed to synthesize complementary DNA (cDNA) using a first-strand cDNA synthesis kit (Takara. Japan). Quantitative assays were performed on each cDNA using the primers listed in Table 3 with SYBR® Premix Ex Taq™ (Takara, Japan) in the given cycling condition (95° C. for 30 seconds, 32 cycles of 95° C. for 5 seconds and 60° C. for 30 seconds), as provided by the manufacturer's instruction.

Wildtype mice with similar weight and age were verified for the presence of the Bmal1 gene prior to habitat in the same environment with a regular day and night cycle (12 hours for each of day and night time durations) for three days. On the fourth day, mice were deeply anesthetized with isoflurane inhalation followed by brain extraction for profiling hour-based Bmal1 expression. This process was repeated for every two hours until 24-hour cycle was completed.

The cerebellar tissue was isolated from anesthetized mice (six weeks old) and fixed with paraformaldehyde in 4% PBS (Thermofisher scientific, USA) and overnight at 4° C. The cerebellar tissue was then dehydrated by being incubated with PBS containing 30% sucrose at 4° C. then embedded in optimal cutting temperature (OCT) medium (Thermofisher scientific, USA) and frozen by using dry ice. The frozen tissues were cut with a cryostat (Thermo HM525NX Cryostat with UV Disinfection, Thermofisher scientific, USA) at 20 pm thickness and slices were placed on glass slides (Thermofisher scientific, USA). For immunohistochemical analysis, cerebellar tissue specimens were washed with PBS for 30 min and blocked with 10% bovine serum albumin (Sigma-Aldrich, USA) in PBST for 30 min at room temperature and then incubated with respective primary antibodies (Calbindin [8 μg/mL]: Invitrogen, USA; Tyrosine Hydrolase [1:400]: Invitrogen. USA) for overnight at 4° C. The specimens were further incubated with secondary antibodies (1:1500, Invitrogen. USA) for overnight at 4° C. The specimens were finalized by incubated with VECTASHIELD which consist of DAPI (Vector laboratories. USA) for 30 minutes then covered with cover slip before being sealed. The DAPI intensity and primary antibody (Calbindin or Tyrosine Hydrolase) intensity were recorded. The relative primary antibody intensity was calculated based on the following formula:

$$Relative primary antibody intensity = \frac{Primary antibody intensity}{DAPI intensity}$$

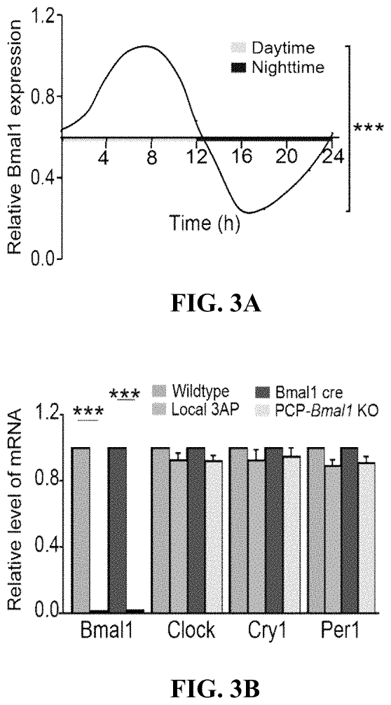

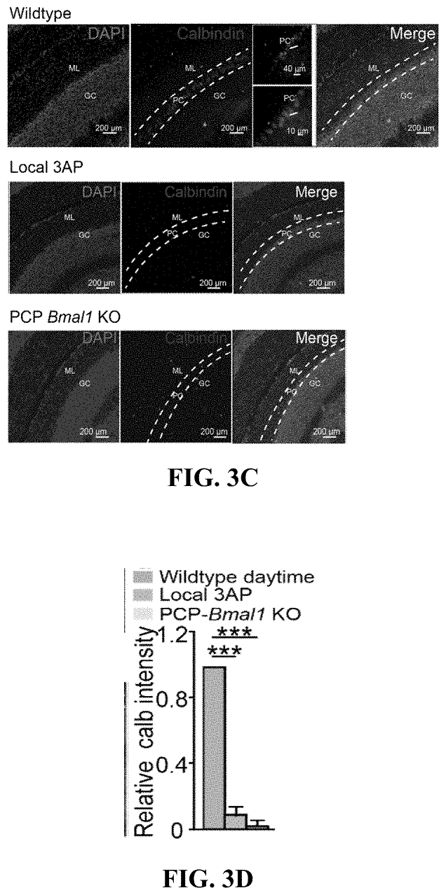

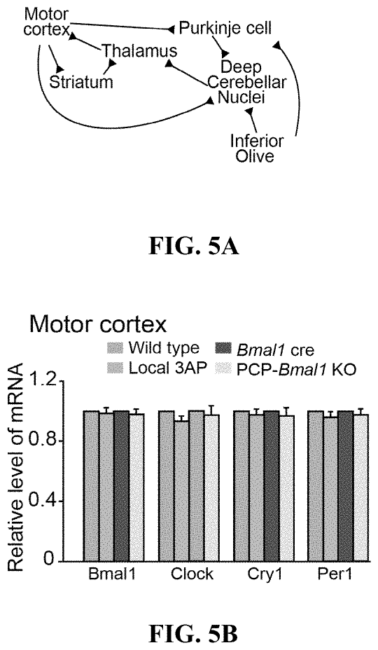

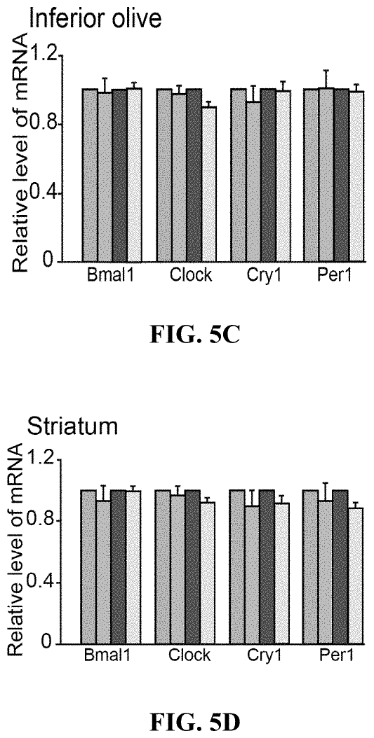

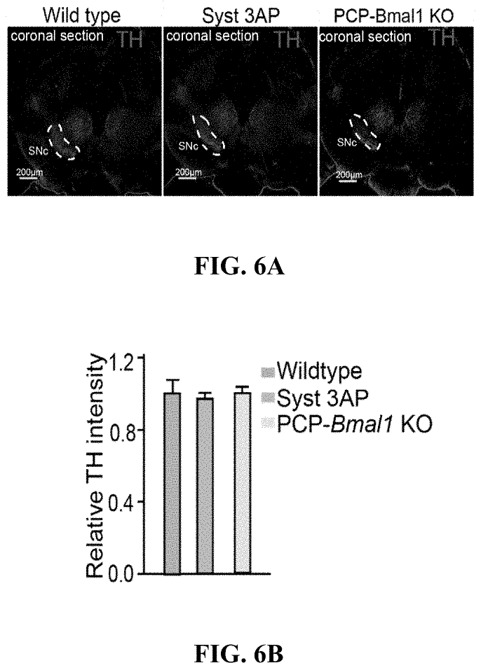

Whether mRNA expression of cerebellar Bmal1 is correlated to ataxia, like the whole-body Bmal1 KO model, several circadian clock genes such as Clock, Cry1, and Per1, but not Bmal1, were shown to be expressed in systematically 3AP-intoxicated mice, specifically in their cerebellum (Table 4). As consistent with the behavioral observation, wildtype mice showed diurnal expression of Bmal1 (FIG. 3A). When the wildtype and Bmal1-cre mice were served as controls for local 3AP-intoxicated and PCP-Bmal1 KO mice, respectively, mRNA expression of Bmal1 was far reduced in the 3AP-intoxicated and PCP-Bmal1 KO mice at daytime, unlike that of other circadian clock genes (FIGS. 3B, 5B-5D; Table 4). The correlation of Bmal1 to ataxia was additionally confirmed with the immunohistochemical method. Calbindin, a molecular marker of Purkinje cells, was used for predicting quantitative expression of Bmal1 in the 3AP-injected and PCP-Bmal1 KO mice, as Bmal1 can promote Ca²⁺ influx which is known to regulate the expression of calbindin (more detail in discussion)^{2,22}. Both chemically-induced and genetically-modified Bmal1-deficient mice showed a low expression of calbindin in the Purkinje cell layer compared to that of wildtype mice (FIG. 3C, WT, 1.00 vs 3AP, 0.123±0.052, n=6, p<0.001, F=26461.7; WT vs PCP-Bmal1 KO, 0.073±0.0342, n=6, p<0.001, F=6361.6). Also, the present ataxia models were associated with cerebellum-dependent ataxia but not substantia nigra-dependent motor abnormality as Tyrosine Hydrolase (TH), a dopaminergic neuronal marker, was similarly expressed in the substandia nigra compacta (SNc) among all three mice groups (FIGS. 6A and 6B, Wildtype vs 3AP ataxia model: p=0.832; Wildtype vs PCP-Bmal1 KO: p=0.896). This result further supports the notion that ataxia is caused by Bmal1 gene deficiency in cerebellar Purkinje cells.

TABLE 4

| | Relative level of mRNA | | | | |
|---|---|---|---|---|---|
| Subject | | Bmal1 | Clock | Cry1 | Per1 |
| 25 mg | Cerebellum | 0.82 | 0.83 | 0.82 | 0.81 |
| 3AP | | (0.102) | (0.129) | (.0114) | (0.156) |
| | Motor | 0.93 | 0.93 | 0.87 | 0.87 |
| | cortex | (0.113) | (0.123) | (0.132) | (0.148) |
| | Inferior | 0.85 | 0.86 | 0.74 | 0.88 |
| | Olive | (0.102) | (0.106) | (0.146) | (0.169) |
| | Striatum | 0.94 | 0.77 | 0.91 | 0.76 |
| | | (0.100) | (0.098) | (0.097) | (0.134) |
| 35 mg | Cerebellum | 0.00*** | 0.94 | 0.66 | 0.77 |
| 3AP | | | (0.165) | (0.103) | (0.144) |
| | Motor | 0.00*** | 0.82 | 0.68 | 0.87 |
| | cortex | | (0.196) | (0.156) | (0.098) |
| | Inferior | 0.00*** | 0.59* | 0.70 | 0.90 |
| | Olive | | (0.065) | (0.135) | (0.087) |
| | Striatum | 0.00*** | 0.87 | 0.89 | 0.86 |
| | | | (0.134) | (0.108) | (0.116) |
| Bmal1 | Cerebellum | 0.00*** | 0.84 | 0.99 | 0.79 |
| KO | | | (0.143) | (0.099) | (0.078) |
| | Motor | 0.00*** | 0.66 | 0.76 | 0.68 |
| | cortex | | (0.079) | (0.134) | (0.122) |
| | Inferior | 0.00*** | 0.54* | 0.84 | 0.65 |
| | Olive | | (0.023) | (0.103) | (0.033) |
| | Striatum | 0.00*** | 0.75 | 0.61* | 0.60* |
| | | | (0.122) | (0.088) | (0.021) |

0.00 = extremely low concentration or the absence of the gene tested;
bracket = standard deviation;
*p-value <0.05;
***p-value <0.001

### EXAMPLE 4—DIMINISHED ATAXIA-LIKE BEHAVIORS BY BMAL1 ADMINISTRATION

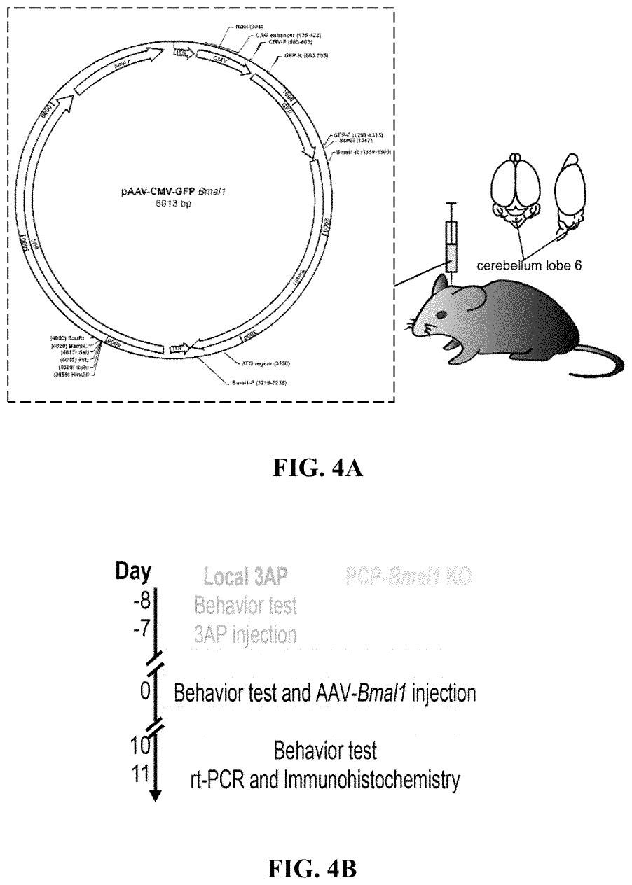

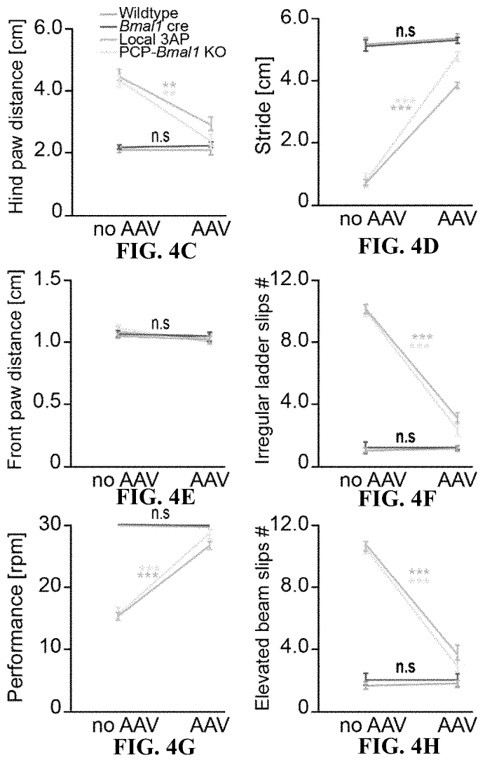

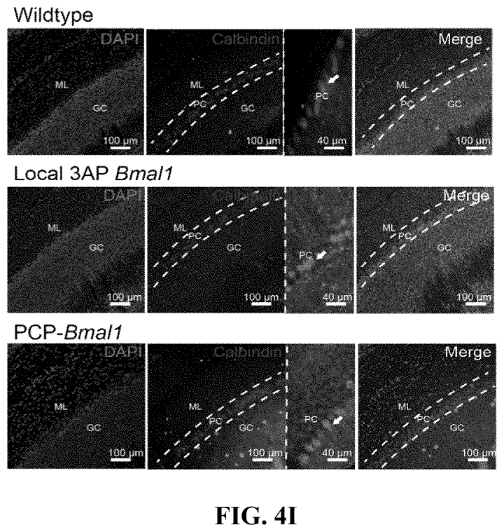

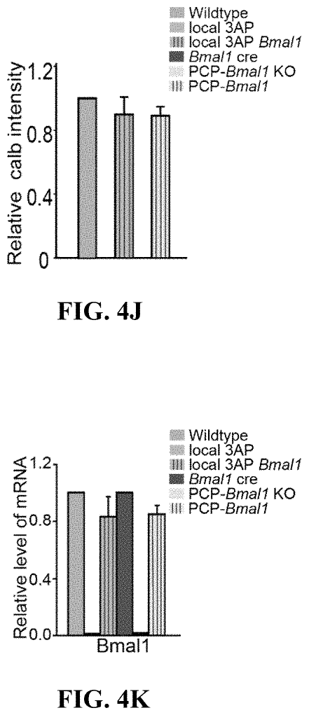

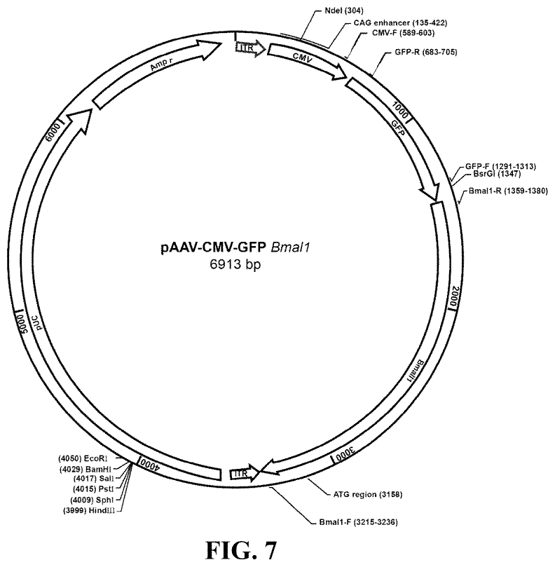

Local injection of a Bmal1-containing AAV was applied to the two ataxia models of local 3AP and PCP-Bmal1 KO mice. Targeted gene, Bmal1 (ARNTL, NM001178.6; SEQ ID NO: 22) was inserted under the CMV promoter with a GFP tag (FIG. 4A and FIG. 7) to generate Bmal1-containing viral vector (pAAV-CMV-GFP Bmal1) (SEQ ID NO: 23). An AAV devoid of Bmal1 was used as the control, 800 nL of 5×10⁴ pfu/mL Bmal1 AAV was loaded to 3.5″ capillary tube (Drummond, USA) and injected to the cerebellum with a coordinate (Bregma, −7.08, 0, −2.1) through Nanoject III (Drummond, USA). Behavior tests were performed on the day 10 after the injection. ANOVA pair t-test was applied to compare between Bmal1-deficient mice and Bmal1 overexpressed mice.

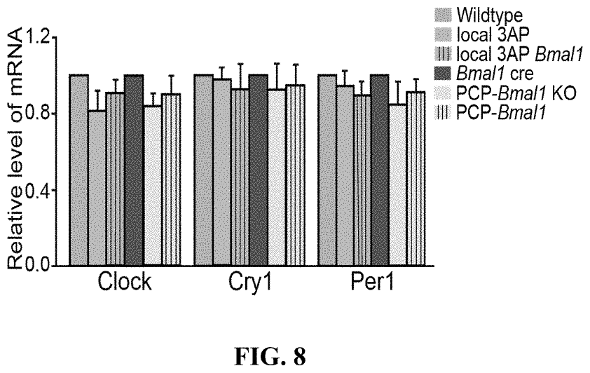

To verify a causal relationship and function of Bmal1 to ataxia in vivo, an AAV of serotype 2 (AAV2) containing Bmal1 gene was injected in the lobe 6 of the cerebellum to compensate for the Bmal1 devoid by 3AP and Bmal1 KO (FIG. 4A). The same behavior tests were performed to determine if the viral administration of Bmal1 by the AAV2 can rescue the motor behaviors impaired by local 3AP and PCP-Bmal1 KO (FIG. 4B for experimental timeline). In the footprint test, to observe 10 days after the viral administration of Bmal1 to local 3AP-intoxicated and PCP-Bmal1 KO mice, there were significant differences in hind paw distance and stride, but not front paw distance (FIG. 4C, hind paw distance, wildtype, 2.13±0.088 cm vs. wiltype-Bmal1, 2.05±0.067 cm, n=6, one-way ANOVA, p=0.372, F=321.332; 3AP, 4.28±0.314 cm vs. 3AP-Bmal1, 2.32±0.186 cm, n=6, p=0.0068. F=26.684; Bmal1-cre, 2.17±0.108 cm vs. Bmal1-cre-Bmal1, 2.28±0.067 cm, n=6, p=0.224, F=47.443; PCP-Bmal1 KO, 4.16±0.122 vs. PCP-Bmal1, 2.35±0.112 cm, n=6, p=0.0014, F=347.882; FIG. 4D, stride, wildtype, 5.02±0.073 cm vs. wiltype-Bmal1, 5.17±0.117 cm, n=6, one-way ANOVA, p=0.232, F=21.442; 3AP, 1.10±0.124 cm vs 0.3AP-Bmal1, 4.13±0.442 cm, n=6, p<0.001. F=2230.181; Bmal1-cre, 5.05±0.118 cm vs. Bmal1-cre-Bmal1, 5.19±0.117 cm, n=6, p=0.124, F=134.765; PCP-Bmal1 KO, 1.16±0.162 vs. PCP-Bmal1, 4.72±0.212 cm, n=6, p<0.001, F=347.882; FIG. 4E, front paw distance, wildtype, 1.12±0.117 cm vs. wiltype-Bmal1, 0.95±0.167 cm, n=6, one-way ANOVA, p=0.063, F=122.336; 3AP, 1.10±0.053 cm vs. 3AP-Bmal1, 0.92±0.257 cm, n=6 p=0.07, F=6.043 Bmal1-cre, 1.08±0.148 cm vs. Bmal1-cre-Bmal1, 0.96±0.117 cm, n=6, p=0.084, F=7.662; PCP-Bmal1 KO, 1.10±0.112 vs. PCP-Bmal1, 0.95±0.123 cm, n=6, p=0.077, F=47.262). Also, Bmal1 administration enhanced performance in the irregular ladder (FIG. 4F, wildtype, 0.87±0.098 vs. wiltype-Bmal1, 0.95±0.103, n=6, one-way ANOVA, p=0.114, F=43.367; 3AP, 11.2±0.075 vs. 3AP-Bmal1, 2.3±0.234, n=6, p<0.001. F=209.958; Bmal1-cre, 0.85±0.098 vs. Bmal1-cre-Bmal1, 0.90±0.108, n=6, p=0.109, F=7.664; PCP-Bmal1 KO, 10.4±0.145 vs. PCP-Bmal1, 2.1±0.234, one-way ANOVA, n=6, p<0.001. F=123.765), rotarod (FIG. 4G, rotarod: wildtype, 30 rpm vs. wiltype-Bmal1, 30 rpm, n=6, one-way ANOVA; 3AP, 14.2±0.130 rpm vs. 3AP-Bmal1, 27.3±0.266 rpm, n=6, p<0.001, F=74.303; Bmal1-cre, 30 vs. Bmal1-cre-Bmal1, 30, n=6; PCP-Bmal1 KO, 14.7±0.336 rpm vs. PCP-Bmal1, 28.3±0.118 rpm, one-way ANOVA, n=6, p<0.001. F=20138.205), and elevated beam tests (FIG. 4H, elevated beam: wildtype, 0.85±0.108 vs. wiltype-Bmal1, 0.90±0.112, n=6, one-way ANOVA, p=0.184, F=512.345; 3AP, 10.7±0.176 vs. 3AP-Bmal1, 2.2±0.321, n=6, p<0.001. F=26.941; Bmal1-cre, 0.90±0.108 vs. Bmal1-cre-Bmal1, 0.95±0.123, n=6, p=0.098, F=32.334; PCP-Bmal1, 2.2±0.177 vs PCP-Bmal1 KO, 10.1±0.118, one-way ANOVA, n=6, p=0028, F=43.312). Calbindins down-expressed by 3AP and Bmal1 KO were restored after Bmal1-containing AAV administration (FIG. 4I and FIG. 4J, Calbindin intensity, WT, 1 vs 3AP-Bmal1, 0.91±0.123, one-way ANOVA, n=3, p=0.224; PCP-Bmal1, 0.87±0.076, p=0.432). Also, the Bmal1 mRNA expression was restored by Bmal1-containing AAV administration while other circadian genes such as Clock, Cry1, and Per1 remained similar (FIG. 4H and FIG. 8). All data are shown as the mean±standard error (SEM). ANOVA test was performed for group comparison (significance, *P<0.05; **P<0.01; ***P<0.001; n.s.: not significant). Pair t-test was applied for before and after AAV-Bmal1 injection (significance, **P<0.01; ***P<0.001; n.s.: not significant). These results confirm that Bmal1 is deeply involved in ataxia manifestation.

In summary, the Bmal1 KO mice prepared according to various embodiments of the present invention show behavior deficits in four motor behavior assays as described herein (footprint, irregular ladder, rotarod, and elevated beam tests). An involvement of Bmal1 in ataxia-like behaviors is identified in three observations: 1) Behavior symptoms of ataxia in the Bmal1-deficient mice, 2) no Bmal1 mRNA expression in the 3AP-intoxicated ataxia model, and 3) Alleviated ataxia by Bmal1 administration. Additionally, the behavioral test results show the dramatic shift of motor activities in the circadian period which are synchronized with the Bmal1 expression pattern in the cerebellum. Bmal1-deficient mice with the impaired shift of motor functions strengthen the correlation of Bmal1 to ataxia.

In spite of human being, motor activities often synchronize with circadian rhythm: Motor skills are heightened during the high expression of Bmal1 usually in the daytime and gradually become worse during the low expression of Bmal1 in the nighttime. This diurnal pattern disappears in patients who experience a severe deficit of motor activities 23.24. Interestingly, it is proven in the present invention that mice known as nocturnal animals have the same circadian rhythm as humans. It can probably be because housed mice are tamed with the conditioned behavior modification^{25,26}. Per1, Cry1, and Clock genes are not as significantly suppressed as Bmal1 in the mRNA expression data obtained in the 3AP-injected ataxia mice. As Bmal1 is closely linked with these circadian clock genes in the regulation of the negative feedback loop²⁷, it is noteworthy that Clock, Cry1, and Per1 genes should not be precluded in studying ataxia of animals and humans.

Although malfunction of Purkinje cells has been widely reported to link with cerebellar ataxia and motor learning, respectively^{28,29}, the present disclosure additionally shows that Purkinje cell-specific Bmal1 deficiency is associated with the decrement of motor skills and learning. Such motor dysfunction is correlated with the dramatic reduction of calbindin expression in the Bmal1-deficient mice. On the other hand, Bmal1 introduction can recover motor behaviors impaired by Bmal1 deficiency, suggesting heavy reliance of Purkinje cells on Bmal1-mediated motor behaviors. This finding is consistent with a previous study that the presence of Bmal1 can activate the RORα transcription factor, a pivotal nuclear receptor for Purkinje cells which mediates expression of Purkinje cells and also promote the Purkinje cell growth in the cerebellum³⁰⁻³³. There are lines of evidence showing the roles of glia on Bmal1-mediated motor behaviors. Bmal1 deficiency can lead to the elevation of the microglial activity^{34,35}. According to some other studies^{36,67}, Bmal1 deficiency can lead to abnormal motor behaviors by the malfunction of glia as well as Purkinje cells.

Calbindin-D28K (or 28 kDa calbindin-D) in Purkinje cells plays an important role in motor control through rapid calcium buffering³⁸, 3AP depletes nicotinamide adenine dinucleotide (NADH, a coenzyme found in all living cells) and disrupts the electron transport at the complex I step, thereby leading to the reduction of Ca²⁺-binding calbindin in Purkinje cells as the disruption of the complex I step causes the uncontrolled changes in Ca²⁺ homeostasis^{39,40,41}. Interestingly, 3AP increases the nicotinic acid, an inhibitor for the DNA-binding activity of Bmal1, which results in the reduction of Bmal1 expression, which is consistent with the result in the present disclosure showing 3AP-induced Bmal1 expression^{42,43}. Meanwhile. Bmal1 plays an essential role as a negative regulator of p38 MAPK pathway that is known to be a Ca²⁺ regulator^{21,22}. Thus, Bmal1 expression can inhibit p38 MAPK signaling, later promoting Ca²⁺ influx^{44,45}. These previous studies underpin the findings in the present disclosure that Bmal1-deficient mice show a lack of calbindin expression which is reversed with additional employment with Bmal1.

The immunohistochemical and mRNA expression data obtained from the Bmal1-deficient animal model after local (e.g., stereotaxic local injection to cerebellum) or systematic administration (e.g., i.p. injection) of the viral vector containing the Bmal1 gene of the present invention also suggest the potential of using a viral vector of similar kind to restore the expression thereof in cerebellum of a subject in need thereof.

Although the invention has been described in terms of certain embodiments, other embodiments apparent to those of ordinary skill in the art are also within the scope of this invention. Accordingly, the scope of the invention is intended to be defined only by the claims which follow.

### REFERENCE

ADDIN EN.REFLIST 1 Ashizawa, T. & Xia, G. Ataxia. Continuum (Minneapolis, Minn.) 22, 1208-1226, doi:10.1212/CON.0000000000000362 (2016).
2 Fogel, B. L. et al. Exome sequencing in the clinical diagnosis of sporadic or familial cerebellar ataxia. JAMA neurology 71, 1237-1246, doi:10.1001/jamaneurol.2014.1944 (2014).
3 Ferri, F. F. Ferri's Clinical Advisor 2020. 2020 edn, Vol. 22 (Elsevier, 2020).
4 Araújo, M. A. d. et al.
5 Jen, J. C. et al. Primary episodic ataxias: diagnosis, pathogenesis and treatment. Brain 130, 2484-2493, doi:10.1093/brain/awm126 (2007).
6 Teive, H. A. G. & Ashizawa, T. Primary and secondary ataxias. Current opinion in neurology 28, 413-422, doi:10.1097/WCO.0000000000000227 (2015).
7 Akbar, U. & Ashizawa, T. Ataxia. Neurologic clinics 33, 225-248, doi:10.1016/j.ncl.2014.09.004 (2015).
8 Pandolfo, M. & Manto, M. Cerebellar and afferent ataxias. Continuum (Minneap Minn) 19, 1312-1343, doi:10.1212/01.con.0000436158.39285.22 (2013).
9 Klockgether, T., Mariotti, C. & Paulson, H. L. Spinocerebellar ataxia. Nat Rev Dis Primers 5, 24, doi:10.1038/s41572-019-0074-3 (2019).
10 Johnathan Cooper-Knock, P. S., Thomas Jenkins. Clinical and Molecular Aspects of Motor Neuron Disease. (Colloquium Life Science, 2013).
11 Yoshimi Okamoto-Uchida, J. I., Jun Hirayama. A molecular link between the circadian clock, DNA damage responses, and oncogene activation. Oncogenes and Carcinogenesis, doi:10.5772/intechopen.81063 (2018).
12 Ehlen, J. C. et al. Bmal1 function in skeletal muscle regulates sleep. eLife 6, e26557, doi:10.7554/eLife.26557 (2017).
13 Menet, J. S., Pescatore, S. & Rosbash, M. CLOCK:BMAL1 is a pioneer-like transcription factor. Genes & development 28, 8-13, doi:10.1101/gad.228536.113 (2014).
14 Moga, M. M. & Moore, R. Y. Organization of neural inputs to the suprachiasmatic nucleus in the rat. J Comp Neurol 389, 508-534, doi:10.1002/(sici)1096-9861(19971222)389:3<508::aid-cnel 1>3.0.co;2-h (1997).
15 Bunger, M. K. et al. Mop3 is an essential component of the master circadian pacemaker in mammals. Cell 103, 1009-1017, doi:10.1016/s0092-8674(00)00205-1 (2000).
16 Hogenesch, J. B., Gu, Y. Z., Jain, S. & Bradfield, C. A. The basic-helix-loop-helix-PAS orphan MOP3 forms transcriptionally active complexes with circadian and hypoxia factors. Proc Natl Acad Sci USA 95, 5474-5479, doi:10.1073/pnas.95.10.5474 (1998).
17 Mohawk, J. A., Green, C. B. & Takahashi, J. S. Central and peripheral circadian clocks in mammals. Annual review of neuroscience 35, 445-462, doi:10.1146/annurev-neuro-060909-153128 (2012).
18 Laposky, A. et al. Deletion of the mammalian circadian clock gene BMAL1/Mop3 alters baseline sleep architecture and the response to sleep deprivation. Sleep 28, 395-409, doi:10.1093/sleep/28.4.395 (2005).
19 Mohammadi, R., Heidari, M. H., Sadeghi, Y., Abdollahifar, M. A. & Aghaei, A. Evaluation of the spatial arrangement of Purkinje cells in ataxic rat's cerebellum after Sertoli cells transplantation. Folia Morphol (Warsz) 77, 194-200, doi:10.5603/FM.a2017.0091 (2018).
20 Wecker, L., Marrero-Rosado, B., Engberg, M. E., Johns, B. E. & Philpot, R. M. 3-Acetylpyridine neurotoxicity in mice. Neurotoxicology 58, 143-152, doi:10.1016/j.neuro.2016.11.010 (2017).
21 Ikeda, M. & Ikeda, M. Bmal1 is an essential regulator for circadian cytosolic Ca²⁺ rhythms in suprachiasmatic nucleus neurons. The Journal of neuroscience: the official journal of the Society for Neuroscience 34, 12029-12038, doi:10.1523/JNEUROSCI.5158-13.2014 (2014).
22 Chen, G. et al. Circadian Rhythm Protein Bmal1 Modulates Cartilage Gene Expression in Temporomandibular Joint Osteoarthritis via the MAPK/ERK Pathway. Frontiers in Pharmacology 11, doi:10.3389/fphar.2020.527744 (2020).
23 Lieberman, H. R., Wurtman, J. J. & Teicher, M. H. Circadian rhythms of activity in healthy young and elderly humans. Neurobiol Aging 10, 259-265, doi:10.1016/0197-4580(89)90060-2 (1989).
24 van Hilten, J. J., Middelkoop, H. A., Kerkhof, G. A. & Roos, R. A. A new approach in the assessment of motor activity in Parkinson's disease. J Neurol Neurosurg Psychiatry 54, 976-979, doi:10.1136/jnnp.54.11.976 (1991).
25 Sundberg, J. P. & Schofield, P. N. Living inside the box: environmental effects on mouse models of human disease. Dis Model Mech 11, dmm035360, doi:10.1242/dmm.035360 (2018).
26 Bronson, F. H. The adaptability of the house mouse. Sci Am 250, 116-125, doi:10.1038/scientificamerican0384-116 (1984).
27 Bushart, D. D., Chopra, R., Singh, V., Murphy, G. G. & Wulff, H. Targeting potassium channels to treat cerebellar ataxia. 5, 297-314, doi:10.1002/acn3.527 (2018).
28 Nguyen-Vu, T. D. et al. Cerebellar Purkinje cell activity drives motor learning. Nat Neurosci 16, 1734-1736, doi:10.1038/nn.3576 (2013).
29 Sausbier, M. et al. Cerebellar ataxia and Purkinje cell dysfunction caused by Ca2+-activated K+ channel deficiency. Proc Natl Acad Sci USA 101, 9474-9478, doi:10.1073/pnas.0401702101 (2004).
30 Chen, X. R. et al. Mature Purkinje Cells Require the Retinoic Acid-Related Orphan Receptor-α (RORα) to Maintain Climbing Fiber Mono-Innervation and Other Adult Characteristics. The Journal of Neuroscience 33, 9546-9562, doi:10.1523/jneurosci.2977-12.2013 (2013).
31 Kondratova, A. A. & Kondratov, R. V. in Inflammation, Advancing Age and Nutrition (eds Irfan Rahman & Debasis Bagchi) 145-155 (Academic Press, 2014).
32 Serra, H. G. et al. RORα-Mediated Purkinje Cell Development Determines Disease Severity in Adult SCAl Mice. Cell 127, 697-708, doi:https://doi.org/10.1016/j.cell.2006.09.036 (2006).
33 Turek, F. W. & Vitatema, M. H. in Handbook of Clinical Neurology Vol. 99 (eds Pasquale Montagna & Sudhansu Chokroverty) 951-961 (Elsevier, 2011).
34 Wang, X. L. et al. Deficiency of the Circadian Clock Gene Bmal1 Reduces Microglial Immunometabolism. Front Immunol 11, 586399, doi:10.3389/fimmu.2020.586399 (2020).
35 Marín-Teva, J. L. et al. Microglia promote the death of developing Purkinje cells. Neuron 41, 535-547, doi:10.1016/s0896-6273(04)00069-8 (2004).
36 Barca-Mayo, O. et al. Astrocyte deletion of Bmal1 alters daily locomotor activity and cognitive functions via GABA signalling. Nature Communications 8, 14336, doi:10.1038/ncomms14336 (2017).
37 Bellamy, T. C. Interactions between Purkinje neurones and Bergmann glia. Cerebellum 5, 116-126, doi:10.1080/14734220600724569 (2006).
38 Barski, J. J. et al. Calbindin in cerebellar Purkinje cells is a critical determinant of the precision of motor coordination. J Neurosci 23, 3469-3477, doi:10.1523/jneurosci.23-08-03469.2003 (2003).
39 Desclin, J. C. & Escubi, J. Effects of 3-acetylpyridine on the central nervous system of the rat, as demonstrated by silver methods. Brain Res 77, 349-364, doi:10.1016/0006-8993(74)90627-1 (1974).
40 Phillips, R. G. et al. Calbindin D28K gene transfer via herpes simplex virus amplicon vector decreases hippocampal damage in vivo following neurotoxic insults. J Neurochem 73, 1200-1205, doi:10.1046/j.1471-4159.1999.0731200.x (1999).
41 Sharma, L. K., Lu, J. & Bai, Y. Mitochondrial respiratory complex I: structure, function and implication in human diseases. Curr Med Chem 16, 1266-1277, doi:10.2174/092986709787846578 (2009).
42 Beher, W. T. & Anthony, W. L. Studies of antimetabolites. III. The metabolism of 3-acetylpyridine in vivo. J Biol Chem 203, 895-898 (1953).
43 Yoshii, K., Ishijima, S. & Sagami, I. Effects of NAD(P)H and its derivatives on the DNA-binding activity of NPAS2, a mammalian circadian transcription factor. Biochem Biophys Res Commun 437, 386-391, doi:10.1016/j.bbrc.2013.06.086 (2013).
44 Li, B. et al. The circadian clock regulator Bmal1 affects traumatic brain injury in rats through the p38 MAPK signalling pathway. Brain Res Bull 178, 17-28, doi:10.1016/j.brainresbull.2021.11.003 (2022).
Wilk-Blaszczak, M. A. et al. The Mitogen-Activated Protein Kinase p38-2 Is Necessary for the Inhibition of N-Type Calcium Current by Bradykinin. The Journal of Neuroscience 18, 112, doi:10.1523/JNEUROSCI.18-01-00112.1998 (1998).

What is claimed is:

1. A non-human animal model having a Purkinje cell-specific gene deficiency leading to ataxia, the Purkinje cell-specific gene being one of the circadian clock genes relating to regulation of circadian rhythm, motor skills and learning ability of a subject.

2. The non-human animal model of claim 1, wherein the Purkinje cell-specific gene is brain and muscle ARNT-like protein 1 (Bmal1) gene.

3. The non-human animal of claim 1, wherein the subject is human.

4. A method of screening compounds or molecules that are capable to restore circadian rhythm, motor skills and learning ability in a subject whose loss of the circadian rhythm, motor skills and learning ability is due to a Purkinje cell-specific gene deficiency, the method comprising:
providing the animal model of claim 1 comprising a knockdown of the Purkinje cell-specific gene being one of the circadian clock genes relating to regulation of the circadian rhythm, motor skills and learning ability of the subject;
introducing a composition that is known to be capable to restore the circadian rhythm, motor skills and learning ability of the subject into a first population of the animal model;
introducing the compounds or molecules into a second population of the animal model;
obtaining a sample from each of the first population and the second population of the animal model; and
comparing expression level of the Purkinje cell-specific gene or a biomarker thereof in the sample of the first population with that in the sample of the second population,
if the expression level of the Purkinje cell-specific gene or the biomarker thereof in the second population being comparable to that in the first population, the compounds or molecules being determined to be capable to restore the circadian rhythm, motor skills and learning ability of the subject;
the composition containing the Purkinje cell-specific gene and being capable to restore expression of the Purkinje cell-specific gene specifically in Purkinje cells and cerebellum of the subject.

5. The method of claim 4, wherein the composition is introduced either locally or systematically into the animal model.

6. The method of claim 4, wherein the composition comprises a viral vector containing the Purkinje cell-specific gene.

7. The method of claim 6, wherein the viral vector is selected from adeno-associated virus.

8. The method of claim 4, further comprising performing motor skill and learning ability related behavioral tests on the first and second populations of the animal model before and after being respectively introduced with the composition and the compounds or molecules, and comparing the behavioral test results with those obtained from animals of the same species as the animal model but without the Purkinje cell-specific gene deficiency.

9. The method of claim 8, wherein the behavioral tests comprise footprint test, irregular ladder test, rotarod test and body balance test.

10. The method of claim 4, wherein the sample from the first and second populations of the animal model comprises cerebellar tissues and at least one type of nucleic acid from the Purkinje cells of the animal model.

11. The method of claim 10, wherein the at least one type of nucleic acid is a total RNA of the Purkinje cells.

12. The method of claim 4, wherein the Purkinje cell-specific gene is brain and muscle ARNT-like protein 1 (Bmal1) gene.

13. The method of claim 12, wherein the biomarker is calbindin.

14. The method of claim 13, wherein the number of Purkinje cells expressing calbindin corresponds to the expression level of Bmal1 gene in the sample.

15. The method of claim 4, wherein the subject is human.

16. A method for making the non-human animal model of claim 2, comprising:
crossbreeding a target mouse having a locus with a cell-specific Cre recombinase expressed mouse,
the locus containing the Bmal1 gene floxed with at least two loxP sites,
the expressed Cre recombinase specifically targeting Purkinje cells and recognizing the at least two loxP sites to delete the locus containing the Bmal1 gene in the Purkinje cells of the target mouse such that a Purkinje cell-specific Bmal1 knockout (PCP-Bmal1 KO) mouse is generated.

17. A composition comprising a Purkinje cell-specific gene relating to regulation of circadian rhythm, motor skills and learning ability of a subject, the Purkinje cell-specific gene being one of the circadian clock genes and expressed in a higher level at daytime than nighttime.

18. The composition of claim 17, wherein the composition is selected from a nucleic acid or viral vector capable of restoring expression of the Purkinje cell-specific gene in the subject with a deficiency of the Purkinje cell-specific gene after administering the composition locally or systematically to the cerebellum of the subject.

19. The composition of claim 18, wherein the Purkinje cell-specific gene is Bmal1.

20. A method for treating ataxia in a subject in need thereof comprising administering the composition of claim 19 locally or systematically to the cerebellum of the subject.
